(19) United States
(12) Patent Application Publication
(10) Pub. No.: US 2010/0324626 A1
(43) Pub. Date: Dec. 23, 2010

(54) Electrotherapy Stimilator for Osteoarthritis

(75) Inventor: Joshua A. Lefkovitz, Chicago, IL (US)

Correspondence Address: PATENT, COPYRIGHT & TRADEMARK LAW GROUP, 4199 Kinross Lakes Parkway, Suite 275, RICHFIELD, OH, 44286, US

(73) Assignee: Management Technologies, Inc.

(21) Appl. No.: 12/821,357

(22) Filed: Jun. 23, 2010

Related U.S. Application Data
(60) Provisional application No. 61/219,500, filed on Jun. 23, 2009.

(51) Int. Cl. A61N 1/36

(52) U.S. Cl. 607/51

(57) ABSTRACT

An electrically conductive and compressive wrap is provided having at least a liner of fabric that electronically emits infrared heat therapy to aid electrotherapy into a patient. The conductive wrap emits a low-level pulsed electrical signal associated with the treatment of osteoarthritis and rheumatoid arthritis. The low-level pulse is provided at low amplitude where is it not physically perceived by the user, but at a level that closely mimics the electrical signal that occurs in naturally healing cartilage. The conductive silver fabric serves as an electrode that delivers optimal distribution of current around anatomically adapted, interchangeable and wearable therapy pads for interchangeable use with the therapy system of the present invention. Each pad includes an electrical insulative liner that keeps current applied to the desired area, and a compression cover to simultaneously deliver a therapeutic amount of compressive force to the area.

### RELATED APPLICATIONS

The present application claims benefit of U.S. Provisional Application Ser. No. 61/219,500, filed on Jun. 23, 2009 and incorporated by reference as if fully rewritten herein.

### BACKGROUND OF THE INVENTION

1. Field of the Invention

The present invention is related to an osteoarthritis and rheumatory arthritis therapeutic apparatus and, more particularly, to an electrically conductive wrap comprising at least a liner of fabric that electronically emits infrared heat therapy in as well as electrotherapy into a patient's target joint area.

2. Description of the Related Art

Arthritis is a medical condition that affects nearly 46 million adults in the United States. Arthritis conditions effect the muskoskeletal system; arthritis-related joint problems interfere with basic daily tasks as they include pain, stiffness, inflammation and joint cartilage damage. Osteoarthritis is a type of degenerative arthritis that is caused by the breakdown and eventual loss of the cartilage of one or more joints. In that this cartilage serves as a “cushion” between the bones of the joints, as deterioration progresses, bones begin to rub against each other, resulting in damage to the surface of the joint. Pain, swelling and restricted movement are commonly a result, and the condition can eventually result in significant disability.

There are two main treatment for osteoarthritis: non-surgical; and surgical. Non-surgical treatments can include weight loss, exercise and medications such as non-steroidal anti inflammatory drug (NSAID) therapy. Surgical treatments can include total joint replacement.

A search of prior art reveals no patents that teach the claims of the present invention; however, the following references are considered related.

U.S. Pat. No. 5,273,033, issued in the name of Hoffman, describes an electrical stimulator treatment for osteoarthritis. The patent protects, in relevant part, a method of treating osteoarthritis by application of a pulsed voltage of 90 to 110 hertz, applying these electrical impulses via non-invasive conductive electrodes in contact with a patient's skin proximate to said joint, and having the amplitude of said impulses initially being sufficiently high as to be sensed by said patient and then reducing only the amplitude of said impulses to a level that is subsensory as to said patient. The method is continued at the reduced amplitude and with no adjustment to the widths of said impulses for the remainder of a treatment period.

U.S. Pat. No. 5,766,236, issued in the name of Detty et al., discloses an ankle support brace that further incorporates an electrical stimulation mechanism.

U.S. Pat. No. 6,584,359, issued in the name of Motoi, describes and alternating current wave form generator for cosmetic use.

U.S. Pat. No. 5,067,478, issued in the name of Berlant, describes a glove incorporates a TENS electrode.

U.S. Pat. No. 6,512,955, issued in the name of McEnany et al, discloses the electronic controls systems and circuitry for generating high voltage, electrical stimulation for therapeutic treatment.

U.S. Pat. No. 7,354,748, issued in the name of Brighton, discloses a method for applying electrical signals and/or an electromagnetic field to a patient's knee for treatment of osteoarthritis.

U.S. Pat. No. 7,215,995, issued in the name of Brighton et al, discloses a method for determining the voltage and current required for the application of specific therapeutic application of electromagnetic stimulation to diseased cartilage.

U.S. Pat. No. 7,130,692, issued in the name of Brighten et al, discloses a portable device for applying electromagnetic therapy to a patient's knee.

U.S. Pat. No. 7,179,217, issued in the name of Goodwin et al, discloses a sleeve for applying an electrically conductive coil to a limb for applying electrical stimulation current thereto.

International Patent Publication WO2007/056474, published in the name of Bionicare Medical Technologies, describes a system of multilayer treatment electrodes incorporated into various wraps for arms, hands, etc.

U.S. Patent Publication U.S. 2008/0039901, published in the name of Kronberg et al., discloses a method for modulating the behavior of, and promote the proliferation of connective tissue growth, with electrically stimulating waveforms.

U.S. Patent Publication U.S. 2007/0156180, published in the name of Jaax et al., discloses an implantable electrical stimulation system.

U.S. Patent Publication U.S. 2004/0176805, published in the name of Whelan et al, discloses a system and technique for applying electromagnetic stimulation to tissues in a self contained and portable fashion.

U.S. Patent Publication U.S. 2003/0158585, published in the name of Burnett, discloses an electromagnetic stimulation device incorporating an ergonomic, body contoured appliance.

U.S. Patent Publication U.S. 2002/0193844, published in the name of Michelson et al, describes a miniature, wireless transcutaneous neuron or muscle stimulation unit. The unit is rechargeable or disposable

And, finally, currently pending patent application Ser. No. 11/270,687 is provided which discloses a protective glove with electrical signal interrupt feature.

Consequently, a need exists for a nonsurgical, osteoarthritis therapeutic apparatus that incorporates an electrically conductive compressive wrap naturally emits infrared heat therapy to aid electrotherapy into a patient.

### SUMMARY OF THE INVENTION

The present invention is an electrically conductive and compressive wrap comprising at least a liner of fabric that electronically emits infrared heat therapy to aid electrotherapy into a patient. The conductive wrap emits a low-level pulsed electrical signal associated with the treatment of osteoarthritis and rheumatoid arthritis. The low-level pulse is provided at low amplitude where is it not physically perceived by the user, but at a level that closely mimics the electrical signal that occurs in naturally healing cartilage.

The conductive silver fabric serves as an electrode that delivers optimal distribution of current around anatomically adapted, interchangeable and wearable therapy pads for interchangeable use with the therapy system of the present invention. Each pad includes an electrical insulative liner that keeps current and heat applied to the desired area, and a compression cover to simultaneously deliver a therapeutic amount of compressive force to the area.

Advantages of such an electrically conductive and electronically heated compressive wrap include improved muscle recovery, as well as expedited and targeted improved blood flow for targeted nutrient supply.

### BRIEF DESCRIPTION OF THE DRAWINGS

Advantages and features of the present invention are better understood with reference to the following and more detailed description and claims taken in conjunction with accompanying drawings, in which like elements are identified with like symbols.

FIG. 1 is a perspective view illustrating the electrotherapy stimulator assembly and system according to the preferred embodiment of the present invention;

FIG. 2 is a detailed perspective view of a controller for use therewith;

FIG. 3 is a perspective view illustrating an exemplary therapy pad for use therewith incorporating an anatomically adapted, wearable knee therapy pads for interchangeable use with the therapy system of the present invention;

FIG. 4 is a perspective view illustrating exemplary therapy pads for use therewith incorporating an anatomically adapted, wearable arm therapy pads for interchangeable use with the therapy system of the present invention;

FIG. 5 is a detailed view illustrating exemplary an arm pad element as shown in

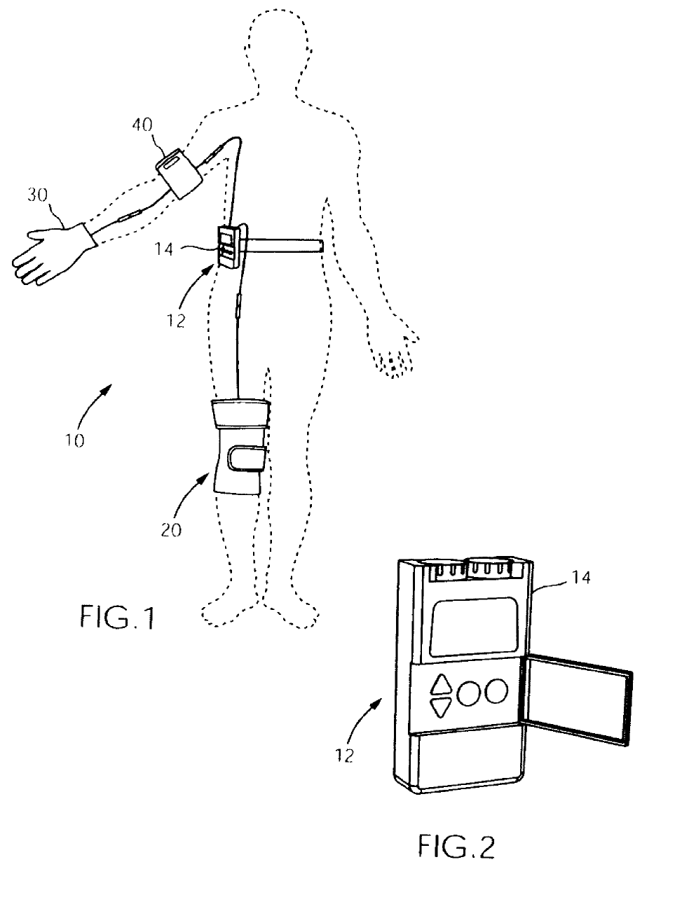

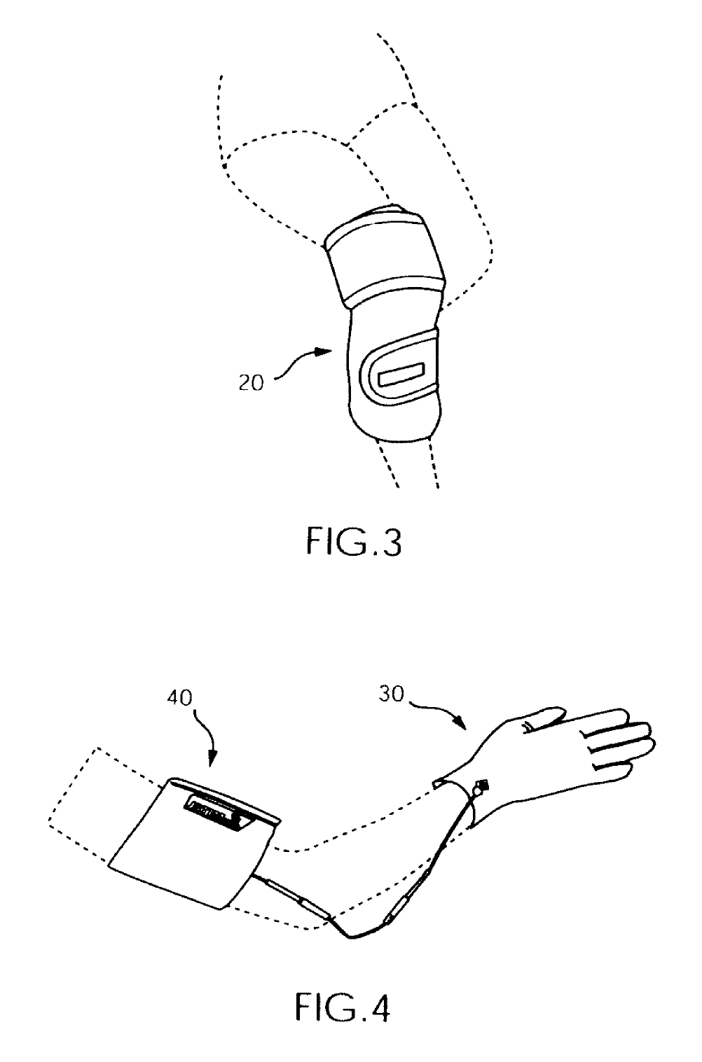

FIG. 4;

FIG. 6 is a detailed view illustrating exemplary an glove or hand pad element as shown in FIG. 4; and

FIG. 7 is a detailed view illustrating exemplary heat wrap element therapy pads for interchangeable use with the therapy system of the present invention.

### DESCRIPTION OF THE PREFERRED EMBODIMENTS

The best mode for carrying out the invention is presented in terms of its preferred embodiment, herein depicted within the Figures.

### 1. Detailed Description

Referring now to the figures, a electrotherapy stimulation assembly and system, generally noted as 10, for implementation of a therapeutic regimen for osteoarthritis is shown. The system 10 includes a joint stimulation electrotherapy device 12 for producing a low-level pulsed electrical signal associated with the treatment of osteoarthritis and rheumatoid arthritis. This small, battery powered electronic housing 14 incorporates the electronics that generates, selectively, two separate signals: the first is a sub-threshold electrical signal that provides a therapeutic regenerative reaction to a joint's cartilage; the second is an otherwise conventional tens signal that provides a relief reaction for rheumatoid arthritis as well as other soft tissue injury.

A soft knee wrap 20 is further provided that is made of comfortable and breathable material. Adapted to fit closely around the knee and thigh of a user, it is intended to be fitted and comfortable in compressive contact with the user's knee.

A soft hand wrap 30 is additionally provided that is made of the same material, adapted to fit like a glove in compressive contact around a user's wrist.

Both the knee wrap 20 and hand wrap 30 are formed of an electrically conductive material and have a wrap a liner of fabric that naturally emits infrared heat therapy to aid electrotherapy into a patient. In a preferred embodiment a silver thread is woven into the liner. The silver thread, when electrically stimulated by the conduction of the therapeutic signal, additionally emits an infrared radiant heat of a sufficient and necessary wavelength as to have a known therapeutic effect to the user. In conjunction with a conformable compressive force, the system can provide a targeted and portable IR therapy system.

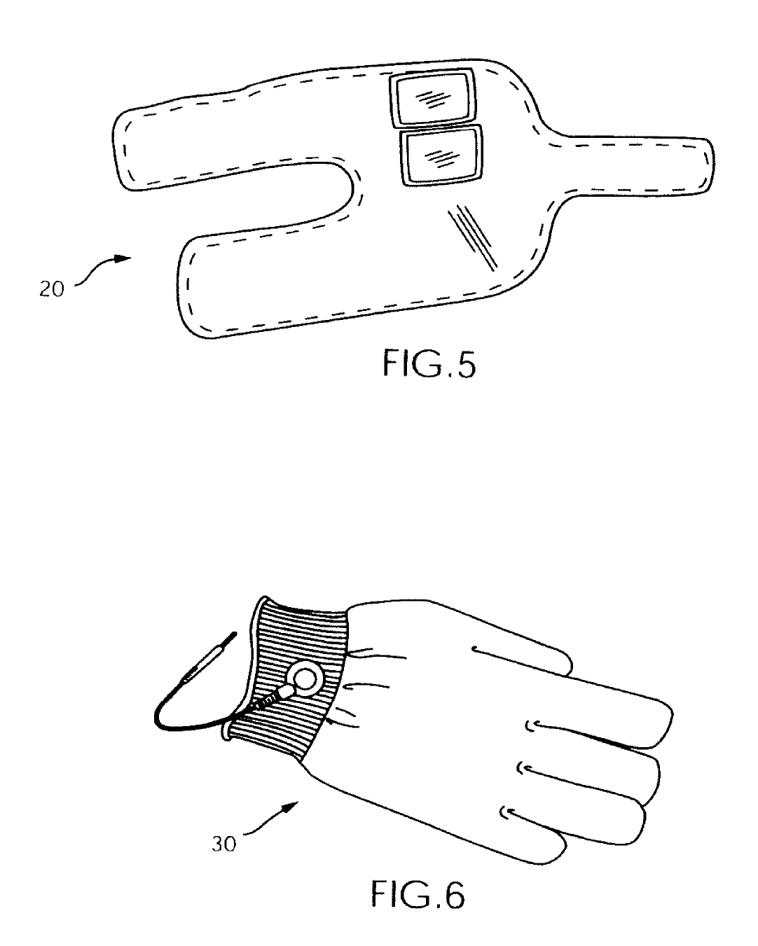

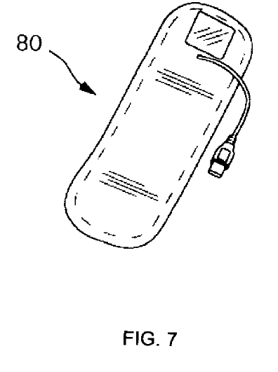

Additional anatomically adapted, wearable therapy pads are also provided for interchangeable use with the therapy system of the present invention and adapted to particular use. By way of example, FIG. 5 is a detailed view illustrating exemplary an arm pad elemen40 t as shown in FIG. 4, and FIG. 6 is a detailed view illustrating exemplary an glove or hand pad element 30 as shown in FIG. 4. FIG. 7 is a detailed view illustrating conductive heat wrap element ion an exemplary therapy pad 80, all for interchangeable use with the therapy system of the present invention.

The stimulator circuit is anticipated as provided dual, isolated channels capable of operating in two distinct modes. The first mode provides an voltage output range of between 0-40 volts peak to peak with a current output range 0-80 mA peak at 500 ohms resistive load. A pulse amplitude of 0 to 40V is generated at a frequence profile of 95 Hz±5 Hz for 30 minutes, followed by 80 Hz±5 Hz for 30 minutes. An auto shut off occurs after 60 minutes. Further, a voltage pulse width of 250 μS±20 μS is provided, with a wave form having symmetrical biphasic square wave characteristics.

Additionally, the second mode provides an voltage output range of between −2 volts ti +15 volts peak to peak with a current output range of −4 mA to 30 mA peak at 500 ohms resistive load, with a 0.2 mA average for a positive spike at 500 ohms resistive load. A pulse frequency capability is further 85 Hz±5 Hz for 25 minute, followed by 80 Hz±5 Hz for 55 minutes, followed by 75 Hz±5 Hz for 25 minutes. This pulse frequency cycle is anticipated as being repeated 4 times per 7 hour treatment block. Further, a voltage pulse width of 1.8 ms±0.2 mS @ 10% pt. of peak, with 0.6 ms±0.2 mS @ 50% pt. of peak. A wave form of spike shaped characteristics is digitally generated.

The foregoing descriptions of specific embodiments of the present invention are presented for purposes of illustration and description only. They are not intended to be exhaustive or to limit the invention to the precise forms disclosed and, obviously, many modifications and variations are possible in light of the above teaching. The embodiments are chosen and described in order to best explain the principles of the invention and its practical application, to thereby enable others skilled in the art to best utilize the invention and the embodiments with various modifications as are suited to the particular use contemplated. It is intended that a scope of the invention be defined by the Claims appended hereto and to their equivalents. Therefore, the scope of the invention is to be limited only by the following claims.

Having thus described the invention what is claimed as new and desired to be secured by Letters Patent is as follows:

1. An electrotherapy stimulator comprising:
an electrically conductive wrap comprising at least a liner of fabric that electronically emits infrared heat therapy to aid electrotherapy into a patient; and
a joint stimulation electrotherapy device for producing a low-level pulsed electrical signal associated with the treatment of osteoarthritis and rheumatoid arthritis having a battery powered electronic housing incorporating electronics that generate a sub-threshold electrical signal that provides a therapeutic regenerative reaction to a joint's cartilage.

2. The electrotherapy stimulator device of claim 1, wherein said electronics further generate a tens signal that provides a relief reaction for rheumatoid arthritis as well as other soft tissue injury.

3. The electrotherapy stimulator device of claim 1, wherein said electrically conductive wrap comprises a plurality of an anatomically adapted, wearable therapy pads for interchangeable use.

4. The electrotherapy stimulator device of claim 3, wherein said plurality of an anatomically adapted, wearable therapy pads for interchangeable use are selected from the group comprising: a soft knee wrap adapted to fit closely around the knee and thigh of a user; a soft hand wrap provided that is adapted to fit like a glove in compressive contact around a user's wrist; and an arm pad element therapy pad.

5. The electrotherapy stimulator device of claim 3, wherein plurality of an anatomically adapted, wearable therapy pads for interchangeable use are formed of an electrically conductive material and have a wrap a liner of fabric that electronically emits infrared heat therapy to aid electrotherapy into a patient.

6. The electrotherapy stimulator device of claim 5, wherein a silver thread is woven into said liner, said silver thread, when electrically stimulated by the conduction of the therapeutic signal, additionally emits an infrared radiant heat of a sufficient and necessary wavelength as to have a known therapeutic effect to the user.

7. The electrotherapy stimulator device of claim 6, wherein said therapeutic signal is adapted for proving an osteoarthritis therapeutic result.

8. The electrotherapy stimulator device of claim 7, wherein said simulator circuit provides a first mode comprising:
an voltage output range of between 0-40 volts peak to peak with a current output range 0-80 mA peak at 500 ohms resistive load;
a pulse amplitude of 0 to 40V is generated at a frequency profile of 95 Hz±5 Hz for 30 minutes, followed by 80 Hz±5 Hz for 30 minutes; and
an auto shut off which occurs after 60 minutes;
wherein a voltage pulse width of 250 μS±20 μS is provided, with a wave form having symmetrical biphasic square wave characteristics.

9. The electrotherapy stimulator device of claim 7, wherein said stimulator circuit further comprises a second mode comprising:
an voltage output range of between −2 volts to +15 volts peak to peak with a current output range of −4 mA to 30 mA peak at 500 ohms resistive load;
a 0.2 mA average for a positive spike at 500 ohms resistive load;
a pulse frequency capability is further 85 Hz±5 Hz for 25 minute, followed by 80 Hz±5 Hz for 55 minutes, followed by 75 Hz±5 Hz for 25 minutes; and
a voltage pulse width of 1.8 ms±0.2 mS @ 10% pt. of peak, with 0.6 ms t 0.2 mS @ 50% pt. of peak;
wherein said pulse frequency cycle can be adapted to being repeated 4 times per 7 hour treatment block.

10. The electrotherapy stimulator device of claim 7, wherein a wave form of spike shaped characteristics is digitally generated.

11. The electrotherapy stimulator device of claim 1, wherein said electrically conductive wraps are in electrical communication with said joint stimulation electrotherapy device via a mini-USB connector/lead wires.

12. A method for providing electrotherapy stimulation to a targeted anatomical site comprising:
a. providing a wearable electrically conductive wrap adapted to conform generally to said targeted anatomical site;
b. providing heat therapy via said electrically conductive wrap; and
c. in combination, providing a low-level pulsed electrical signal at said targeted anatomical site from a joint stimulation electrotherapy device.

13. The method of claim 12, wherein said joint stimulation electrotherapy device produces a low-level pulsed electrical signal associated with the treatment of osteoarthritis and rheumatoid arthritis having a battery powered electronic housing incorporating electronics that generate a sub-threshold electrical signal that provides a therapeutic regenerative reaction to a joint's cartilage.

14. The method of claim 12, wherein said joint stimulation electrotherapy device produces a tens signal that provides a relief reaction for rheumatoid arthritis as well as other soft tissue injury.

15. The method of claim 12, wherein said wearable electrically conductive is selected from a plurality of an anatomically adapted, wearable therapy pads for interchangeable use are selected from the group comprising: a soft knee wrap adapted to fit closely around the knee and thigh of a user; a soft hand wrap provided that is adapted to fit like a glove in compressive contact around a user's wrist; an arm pad element therapy pad; a body wrap element therapy pad; and a wrist wrap element therapy pads.

16. The method of claim 12, wherein said low-level pulsed electrical signal provides a first mode comprising:
an voltage output range of between 0-40 volts peak to peak with a current output range 0-80 mA peak at 500 ohms resistive load;
a pulse amplitude of 0 to 40V is generated at a frequency profile of 95 Hz±5 Hz for 30 minutes, followed by 80 Hz±5 Hz for 30 minutes; and
an auto shut off which occurs after 60 minutes;
wherein a voltage pulse width of 250 μS±20 μS is provided, with a wave form having symmetrical biphasic square wave characteristics.

17. The method of claim 12, wherein said low-level pulsed electrical provides a second mode comprising:
an voltage output range of between −2 volts to +15 volts peak to peak with a current output range of −4 mA to 30 mA peak at 500 ohms resistive load;
a 0.2 mA average for a positive spike at 500 ohms resistive load;
a pulse frequency capability is further 85 Hz±5 Hz for 25 minute, followed by 80 Hz±5 Hz for 55 minutes, followed by 75 Hz±5 Hz for 25 minutes; and
a voltage pulse width of 1.8 ms±0.2 mS @ 10% pt. of peak, with 0.6 ms±0.2 mS @50% pt. of peak;
wherein said pulse frequency cycle can be adapted to being repeated 4 times per 7 hour treatment block.

18. The method of claim 16, wherein said low-level pulsed electrical signal further provides a second mode comprising:
an voltage output range of between −2 volts to +15 volts peak to peak with a current output range of −4 mA to 30 mA peak at 500 ohms resistive load;
a 0.2 mA average for a positive spike at 500 ohms resistive load;
a pulse frequency capability is further 85 Hz±5 Hz for 25 minute, followed by 80 Hz±5 Hz for 55 minutes, followed by 75 Hz±5 Hz for 25 minutes; and
a voltage pulse width of 1.8 ms±0.2 mS @ 10% pt. of peak, with 0.6 ms±0.2 mS @ 50% pt. of peak;
wherein said pulse frequency cycle can be adapted to being repeated 4 times per 7 hour treatment block.

19. The method of claim 17, wherein a wave form is of spike shaped characteristic that is digitally generated.
